(19) United States
(12) Patent Application Publication
(10) Pub. No.: US 2011/0098590 A1
(43) Pub. Date: Apr. 28, 2011

(54) METHODS AND APPARATUSES FOR DETECTING ANALYTES

(75) Inventors: Ian Garbutt, Camas, WA (US); David Urman, Portland, OR (US); John Hunt, Madison, VA (US); Stephen H. Mastin, Gresham, OR (US); Wes Spiegel, Richardson, TX (US); Chris Marsh, Lake Oswego, OR (US)

(73) Assignee: Pulse Health LLC

(21) Appl. No.: 12/912,526

(22) Filed: Oct. 26, 2010

Related U.S. Application Data
(60) Provisional application No. 61/255,027, filed on Oct. 26, 2009.
(60) Provisional application No. 61/255,034, filed on Oct. 26, 2009.

(51) Int. Cl. A61B 5/08

(52) U.S. Cl. 600/532

(57) ABSTRACT

An apparatus for measuring a quantity of an analyte, such as an aldehyde, contained in a breath sample includes a breath collection device and a measurement device. The breath collection device includes a breath inlet area, a breath outlet area, and a reaction chamber. The reaction chamber can include a reagent that is colorimetrically reactive with one or more aldehydes. The measurement device includes a light emitting device and a light measuring device and is configured to provide a quantitative value indicative of the amount of aldehydes present in the breath sample.

### CROSS-REFERENCE TO RELATED APPLICATIONS

This application claims the benefit of U.S. Provisional Application No. 61/255,027, filed on Oct. 26, 2009, and U.S. Provisional Application No. 61/255,034, filed on Oct. 26, 2009, both of which are incorporated herein by reference in their entirety.

### FIELD

The disclosure pertains to apparatuses and methods for collecting and analyzing breath samples to detect the presence of various substances, including those that are related to or indicative of physical conditions or diseases.

### BACKGROUND

Various diagnostic screening and testing methods are available to identify or quantify a medical or physical condition of an individual. Generally, these methods require the collection of a fluid sample (e.g., blood, plasma, and urine) from a patient and the submission of that fluid sample to a laboratory for analysis. For example, there are diagnostic tests available for the quantification of the end products associated with lipid peroxidation. Lipid peroxidation is the process whereby free radicals cause cell damage in the body by removing electrons from lipids in cell membranes. Free radicals are often associated with the consumption of processed foods, alcohol, and the use of tobacco products, and have been implicated as a potential cause or aggravating factor in numerous disease processes. It is also commonly believed that organisms age, at least in part, because cells in the body accumulate free radical damage over time.

Conventional diagnostic tests for lipid peroxidation typically require the collection of a blood, plasma, or urine sample from a patient. Such conventional diagnostic tests are somewhat undesirable, however, since they require the collection of a sample in a relatively invasive manner from the patient. Moreover, such conventional diagnostic tests can be expensive and time-consuming, since they typically involve labor-intensive laboratory analysis of the collected samples.

Testing methods that are based on breath samples are particularly desirable since, unlike blood, urine, or other physical samples, breath samples can be easily obtained from an individual in a simple and non-invasive manner. For example, U.S. Pat. No. 7,285,246, which is incorporated herein by reference in its entirety, discloses a hand-held fluid analyzer for detecting alcohol or other preselected substances in the fluids present in the exhaled breath of a test subject. The '246 patent relies on visual inspection of an indicator reagent to determine whether the preselected substance is present and, as a result, is limited in its ability to detect specific amounts or ranges of a preselected substance in the sample.

### SUMMARY

In some embodiments, the methods and devices are configured to measure target analytes in a patient's breath. The target analytes can include markers of free-radical activity, such as aldehydes. Aldehydes are byproducts of and directly correlated to oxidative stress (also known as free radical damage), along with various associated health risks. Accordingly, the identification and monitoring of aldehyde levels in a patient's breath can provide an indication of health, as well as a benchmark from which a patient can seek improvement. The disclosed methods and devices permit rapid, accurate and convenient assessment of an individual's level of oxidative stress in a clinical or non-clinical setting.

In one embodiment, an apparatus for measuring a quantity of an analyte contained in a breath sample is provided. The apparatus includes a breath collection device and a measurement device. The breath collection device has a breath inlet area, a breath outlet area, and a reaction chamber. The reaction chamber includes a reagent that is colorimetrically reactive with the analyte to be measured. The measurement device includes a light emitting device and a light measuring device. The measurement device can be configured to receive the breath collection device so that the reagent is positioned to receive light from the light emitting device and reflect at least a portion of the received light to the light measuring device.

In specific implementations, the analyte that is measured includes one or more aldehydes. The reagent can be in a solid phase and can include a reagent, such as a Schiff reagent and a silica material. In specific implementations, a Schiff reagent can include rosaniline or pararosaniline.

In other specific implementations, the light emitting device can include a plurality of LEDs. The plurality of LEDs can emit light at the same wavelength or at different wavelengths. In other specific implementations, a first LED can emit light in a range reflected and/or absorbed by the reagent that corresponds, at least in part, to a reactivity of the reagent to moisture and a second LED can emit light in a range absorbed by the reagent that corresponds, at least in part, to a reactivity of the reagent to the analyte.

In other specific implementations, a numeric value generating device can be provided. The value generating device can include an algorithm that generates a quantitative value based on one or more colorimetric measurements taken by the measurement device on the reagent. In other specific implementations, a moisture reactivity measuring device can be provided and the algorithm can be configured to normalize the colorimetric measurements based on a measurement of the amount of moisture captured and/or contained by the reagent. In specific implementations, the moisture reactivity measuring device can include an LED that emits light in a range absorbed by the reagent that corresponds, at least in part, to a reactivity of the reagent to moisture.

In other embodiments, a breath collection device is provided. The breath collection device can include a tubular outer member having a first end portion and a second end portion, a breath inlet area having a first porous plug member positioned in the tubular outer member at the first end portion, a breath outlet area having a second porous plug member positioned in the tubular outer member at the second end portion, and a reaction chamber within the tubular outer member and located between the first and second porous plug members. The reaction chamber can include a reagent that is colorimetrically reactive with one or more substances contained in the exhaled breath of a test subject. The second porous plug member can have a first cylindrical portion having a first diameter and a second cylindrical portion having a second diameter, with the second diameter being smaller than the first diameter. The second cylindrical portion can extend from the first cylindrical portion towards the first porous plug member.

In specific implementations, the breath collection device can also include a breakable container that, at least initially, contains the reagent. The container can also include a wrap extending around at least a portion of the container to reduce the amount of broken pieces of container that result when the container is broken and the reagent released. In specific implementations, the wrap can extend around at least 70% of the container, and more preferably, between about 85% and 95% of the container.

In specific implementations, the device can include a hood member comprising a breath receiving portion and a breath outflow portion. The breath outflow portion can be configured to be removably coupled to the first end portion of the tubular outer member. The breath receiving portion and the breath outflow portion can be configured at an angle of about 70 and 110 degrees relative to one another.

In other embodiments, methods are provided for manufacturing a solid phase reagent for detecting the presence of aldehydes in a breath sample. The methods can include activating a surface of a silica material by lowering the pH of the silica; heating the activated silica material; adding a solution containing a Schiff reagent to the activated silica material; and drying the mixture of the Schiff reagent and the activated silica material.

In specific implementations, the act of activating the surface of the silica material can include adding an acid to the silica material at a ratio of about 1:2 by weight. The acid can be, for example, a phosphoric acid solution. The act of heating the activated silica material can include heating the activated silica material at a temperature greater than about 60 degrees Celsius. The act of adding a solution containing a Schiff reagent to the activated silica material can include adding the Schiff reagent to the activated silica material at a ratio of about 2:1 by weight. In specific implementations, the Schiff reagent can include rosaniline or pararosaniline or derivatives thereof.

In another embodiment, an apparatus for measuring a quantity of an analyte contained in a breath sample is provided. The apparatus has a breath collection device including a breath inlet area, a breath outlet area, and a reaction chamber. The reaction chamber includes a reagent that is colorimetrically reactive with the analyte. The apparatus also has a measurement device including a light emitting device and a light measuring device. The measurement device is configured to receive the breath collection device so that the reagent is positioned to receive light from the light emitting device and reflect at least a portion of the received light to the light measuring device. The light measuring device is configured to take a plurality of reflectance measurements over a predetermined time period for at least two wavelength regions. The apparatus also has a measurement selection device configured to select one or more measurements taken by the measurement device to determine the quantity of the analyte. The measurement selection device is configured to select one or more measurements based on a determination of a rate of change of reflectance of at least one of the two wavelength regions.

In specific implementations, the two wavelength regions include a first region between about 400 nm and 450 nm and a second region between about 550 nm and 600 nm. In other specific implementations, the rate of change of reflectance is determined at the second region.

The foregoing and other features and advantages of the apparatuses and methods described herein will become more apparent from the following detailed description, which proceeds with reference to the accompanying figures.

### BRIEF DESCRIPTION OF THE DRAWINGS

FIG. 1 is a front perspective view of an embodiment of a breath collection device.

FIG. 2 is an exploded view of the breath collection device of FIG. 1.

FIG. 3A is a front elevational view of an embodiment of a plug member that can be used with a breath collection device.

FIG. 3B is a top view of the plug member of FIG. 3A.

FIG. 3C is a bottom view of the plug member of FIG. 3A.

FIG. 4A is a front elevational view of an embodiment of a plug member that can be used with a breath collection device.

FIG. 4B is a bottom view of the plug member of FIG. 4A.

FIG. 5A is a front elevational view of an embodiment of an outer member that can be used with a breath collection device.

FIG. 5B is a top view of the outer member of FIG. 5A.

FIG. 6 is a front elevational view of an embodiment of a container for containing a reagent.

FIG. 7 is a front elevational view of an embodiment of a breath collection device.

FIG. 8 is front perspective view an embodiment of a measurement device for measuring substances present in exhaled breath.

FIG. 9 is a front perspective view of the measurement device of FIG. 8, shown with an open door member for receiving a sample.

FIG. 10 is an exploded view of the measurement device of FIG. 8.

FIG. 11 is a front perspective view of a portion of the measurement device shown in FIG. 8.

FIG. 12 is an exploded view of the portion of the measurement device shown in FIG. 11.

FIG. 13 is a schematic cross-sectional view of a portion of a measurement device.

FIG. 14 is a front elevational view of a portion of a measurement device.

FIG. 15 is a schematic cross-sectional view of a measurement device.

FIG. 16 is a graph that compares reflectance levels of breath samples and unreacted reagent.

FIG. 17 illustrates a flowchart indicating a “Start/Self Test Cycle” for a measurement device.

FIG. 18 illustrates a flowchart indicating a “Ready/Test Start Cycle” for a measurement device.

FIG. 19 illustrates a flowchart indicating a “Measurement Cycle” for a measurement device.

FIG. 20 illustrates a flowchart indicating a “Shutdown Cycle” for a measurement device.

FIG. 21 illustrates a top view of a hood member and a breath collection device.

FIG. 22 illustrates an exploded view of a hood member and a breath collection device.

FIG. 23 illustrates a top view of a hood member and a breath collection device.

FIG. 24 illustrates a section view of the hood member and breath collection device of FIG. 23 taken along line 24-24.

### DETAILED DESCRIPTION

The following description is exemplary in nature and is not intended to limit the scope, applicability, or configuration of the invention in any way. Various changes to the described embodiment may be made in the function and arrangement of the elements described herein without departing from the scope of the invention.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The systems and methods described below relate to non-invasive testing systems and methods of using such systems to identify the presence of various substances in the exhaled breath of a test subject. The substances can either be detected directly in the exhaled breath or in a condensate thereof. As discussed in more detail below, a testing system generally includes a breath collection device, a reagent contained in the breath collection device that exhibits a colorimetric reaction when exposed to a substance present in exhaled breath, and a measurement device that is capable of quantifying the colorimetric reaction resulting from the interaction of the substance in the exhaled breath with the reagent.

The section headings used herein are for organizational purposes only, and are not to be construed as limiting the subject matter disclosed.

### Breath Collection Device

The breath collection device is configured to capture exhaled breath and expose the captured breath to a reagent contained within the breath collection device. Upon exposure to the exhaled breath, and more particularly, upon exposure to one or more substances present in the exhaled breath, the reagent undergoes a chemical reaction that is measureable and/or quantifiable by a measurement device as described in more detail below. The substance that is being detected may be referred to as an “analyte.”

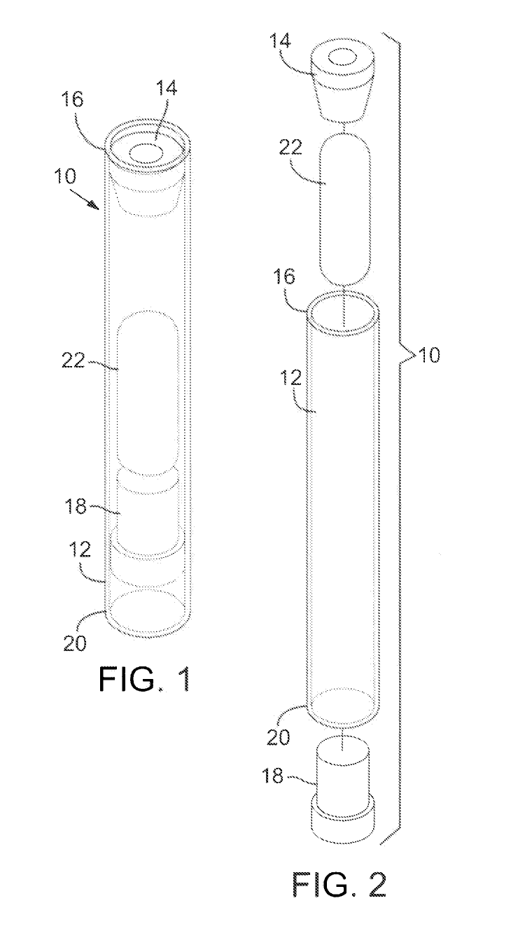

FIGS. 1 and 2 illustrate a perspective view and an exploded view, respectively, of an embodiment of a breath collection device. The breath collection device 10 includes an outer member 12, which, in the illustrated embodiment, is a tubular plastic structure with openings at both ends. A first plug member 14 is received at a first end 16 of the outer member 12 and a second plug member 18 is received in a second end 20 of the outer member 12. The first and second plug members 14, 18 are formed of material that is sufficiently air-permeable to allow a test subject to blow exhaled air through both the first and second plug members. Thus, the first plug member 14 defines a breath inlet area and the second plug member 18 defines a breath outlet area.

A reagent container 22 can be held within the outer member 12 between the first and second plug members 14, 18. The container 22 contains a reagent that is capable of registering a color change when the reagent is exposed to one or more substances present in an exhaled breath of a test subject. To preserve the reagent prior to use, the container 22 preferably holds the reagent in an airtight and/or inert manner. When a test is to be performed, the reagent is released from the container 22 by breaking or otherwise opening the container 22.

In other embodiments, the reagent can be packaged in other ways. For example, the reagent can be packaged in an inert manner by supplying an inert gas into the container that holds the reagent. Alternatively, if the reagent does not need to be contained in an airtight or inert manner prior to use, the reagent may not need to be held in a separate container. For example, the reagent could simply be held between the two plug members 14, 18.

The area between the first and second plug members 14, 18 defines a reaction chamber where the reagent (once released) can interact with the breath sample collected in or blown through the reaction chamber. The reaction chamber is preferably sized to maximize reactivity and reduce the volume of breath required. For example, in one embodiment, the outer member 12 can be about 2.75 inches in length and have an inner diameter of about 0.337 inches. The plug members are preferably less than about a half inch in length and therefore, the length of the reaction chamber is preferably about 1.75 inches or more. In addition, a relatively short/low volume reaction chamber requires a smaller amount of reagent.

In the illustrated embodiment, the container 22 is a glass container (ampoule) that can be broken through the manual application of a compressive force applied to an outer surface of the outer member 12. To facilitate the transfer of the compressive force from the outer member 12 to the container 22, the outer member 12 is preferably formed a material (such as a flexible plastic) that is sufficiently flexible to allow a user to apply a force through the outer member 12 to the container 22 without breaking the outer member 12.

Once the container 22 is broken, the reagent can move freely within the intact outer member 12 and between the plug members 14, 18. Thus, the plug members 14, 18 are preferably sized to prevent reagent from passing around or through the plug members during exhalation (e.g., through the second plug member) or inhalation (e.g., through the first plug member in the event that the test subject mistakenly inhales during testing). Accordingly, the pore size of the plugs can vary depending on the size of the reagent particles. In some embodiments, the pore size of the plugs is preferably between about 100-200 mesh (0.015-0.075 mm). Any clearance between the plugs and the walls of the outer member 12 are also sufficiently tight to substantially avoid inadvertent passage of the reagent past the plugs.

The amount of airflow through the device is based on, at least in part, amount of reagent in the chamber, reagent size, pore size, and pore volume. Pore size and pore volume can be manipulated by using a knowledge of the chemistry of the materials to be used for the plug members. For example, if the plug member is formed of a plastic material, pore size and volume can be varied due to chemical cross linking and/or the selection of different polymers. Accordingly, the threshold amount of pressure required to blow through a device can be increased or decreased by altering one or more of the above-identified variables (e.g., amount of reagent in the chamber, reagent size, pore size, and pore volume).

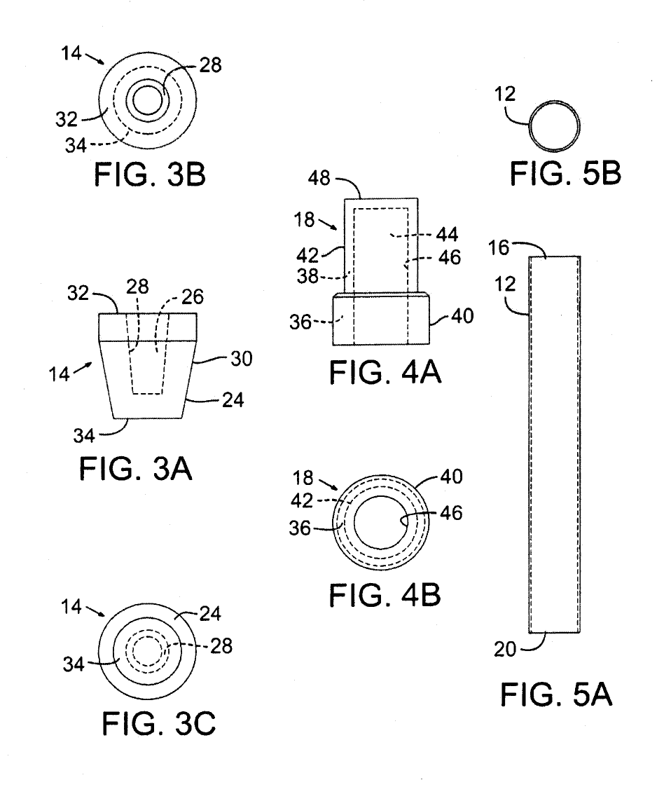

FIG. 3A is a side view of the first plug member 14, FIG. 3B is a top view of the first plug member 14, and FIG. 3C is a bottom view of the first plug member 14. The first plug member 14 can have a tapered section 24, which facilitates placement of the first plug member 14 in the opening of the outer member 12. If desired, first plug member 14 can have a hollow portion 26 that extends partially through a central region of the first plug member 14. The hollow portion 26 can also be tapered to provide a substantially uniform distance between an interior surface 28 and an opposing exterior surface 30 of the first plug member 14. The substantially uniform distance facilitates the passage of air (e.g., exhaled breath) from a first end 32 of the first plug member 14 through the second end 34 and/or the tapered exterior surface 30 of the first plug member 14.

It should be understood that the form of the first plug member 14 can vary. For example, the first plug member 14 can have a shape that is the same as, or similar to, the shape of the second plug member 18.

FIG. 4A is a side view of the second plug member 18 and FIG. 4B is a bottom view of the second plug member 18. Second plug member 18 can include a first cylindrical portion 36 and a second cylindrical portion 38. The first cylindrical portion 36 and the second cylindrical portion 38 can be formed separately and/or of different materials; however, they are preferably integrally formed of the same material. The first cylindrical portion 36 has an exterior surface 40 that has a sufficiently large outer diameter so that the exterior surface 40 is tightly received in the opening of the outer member 12. By providing a tight fit between the exterior surface 40 and the interior surface of the outer member 12, reagent particles can be restricted from passing between the exterior surface 40 and the interior surface of the outer member 12. The second cylindrical portion 38 is preferably in coaxial alignment with the first cylindrical portion 36, with an exterior surface 42 of the second cylindrical portion 38 having a diameter that is smaller than the diameter of the exterior surface 40 of the first cylindrical portion 36.

If desired, to reduce the amount of pressure required to blow through the breath collection device 10, the second plug member 18 can have a hollow portion 44 that extends partially through a central region of the second plug member 18. The hollow portion 44 can be sized to provide a substantially uniform distance between an interior surface 46 and an exterior surface 48 of the second plug member 18. As with the hollow portion of the first plug member described above, the substantially uniform distance between the two surfaces of the second plug member 18 can facilitate the passage of air (e.g., exhaled breath) through the second plug member 18.

As described in more detail below, the measuring device preferably performs a measurement while the outer member 12 is in a vertical orientation with the first plug member 14 at the top and the second plug member 18 at the bottom. In this vertical configuration, reagent particles are at least partially contained in an area (measurement area 64 as shown in FIG. 7) bounded by the interior surface of the outer member 12 and the exterior surface 42 of the second cylindrical portion 38. Thus, as described in more detail below, in the illustrated embodiment, the measurement can be taken with the outer member 12 in any axial orientation because the reagent particles can be distributed in a substantially uniform manner around the exterior surface 42 of the second cylindrical portion 38.

In addition, the collection of exhaled breath is also preferably taken while the breath collection device 10 is a vertical orientation with the first plug member 14 at the top and the second plug member 18 at the bottom. However, because tilting the head can constrict the throat and make it more difficult for a test subject to blow through the breath collection device, it may be desirable to include a hood (cap) member or other such device capable of redirecting air exhaled at a horizontal direction to a vertical (downward) direction. For example, a hood member can comprise a structure that defines a substantially L-shaped pathway capable of redirecting horizontally exhaled breath through the breath collection device while the breath collection device is held in a substantially vertical orientation. The hood member, or other such structure, can be integrally formed with the breath collection device or it can be separately attachable to the breath collection device. In addition, if desired, the hood member, or other such structure, could include an indicator that tells the user whether he or she is blowing with sufficient force. For example, the hood member could include a valve that makes a sound such as a whistle when the user is blowing too hard or too soft.

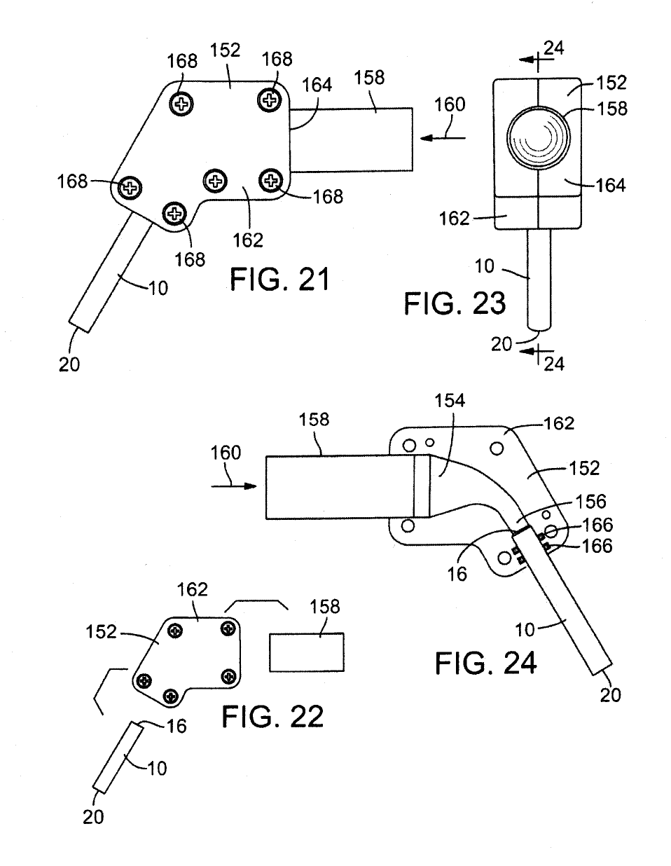

An exemplary embodiment of a hood member is illustrated in FIGS. 21-24. Hood member 150 comprises a main body 152 that has a breath receiving portion (breath inlet) 154 and a breath outflow portion 156. Breath receiving portion 154 is configured to receive a breath sample from a patient. In one embodiment, the breath receiving portion 154 can be coupled to a blow device 158. A patient can blow directly into blow device 158 to deliver breath to the breath receiving portion 154. Blow device 158 can comprise a tube or other similar shape so that a patient can easily cover the device with their mouth to deliver the breath sample to the hood member 150. In a preferred embodiment, the blow device 158 comprises a disposable member, such as a disposable cardboard tube, so that sterile re-use of the hood member 150 can be easily achieved.

Breath outflow portion 156 is configured to deliver the breath sample to the breath collection device 10. Breath collection device 10 can be coupled to the breath outflow portion 156 in such a manner that breath is delivered from the breath outflow portion 156 into the first end 16 of the breath collection device. Thus, as best shown in FIG. 24, breath can be blown into the blow device 158 of the hood member 150 in the direction of arrow 160, pass through an opening in the blow device 158, into the main body 152, and into the breath outflow portion 156. From the breath outflow portion 156, the breath enters the first end 16 of the breath collection device 10. After passing through the plug member, as described in more detail above, the breath collection sample is captured by the breath collection device 10.

The shape of the hood member 150 desirably permits the user to blow in a substantially horizontal direction relative to the ground, while the breath collection device 10 is maintained in a substantially vertical orientation relative to the ground. Thus, the blow device 158 and breath collection device 10 are preferably oriented at approximately 70-110 degrees (e.g., within about 20 degrees from normal or 90 degrees) relative to one another. As shown in FIGS. 21-24, a diameter of the breath device 152 (and/or breath receiving portion 154 can be greater than the diameter of the breath collection device 10 (and/or the breath outflow portion 156). In this manner, a larger volume of breath can be delivered to the breath collection device.

The hood member can be a unitary member or it can comprise a plurality of sections that can be coupled together. For example, as shown in FIGS. 21 and 23, the main body 152 can comprise a top portion 162 and a bottom portion 164. Top and bottom portions 162, 164 can be configured to be substantially mirror image recessed portions so that when they are coupled together (as shown in FIGS. 21 and 23), the recessed portions define an opening that extends from the breath receiving portion 154 to the breath outflow portion 156. The top and bottom portions 162, 164 can be coupled together using a variety of methods, such as one or more screws 168.

In a preferred embodiment, the breath collection device 10 is coupled to the breath outflow portion 156 using one or more resilient members 166. For example, referring to FIG. 24, the resilient members 166 can comprise o-ring members that “cushion” the coupling of breath collection device 10 within the breath outflow portion 156. The cushioning of the breath collection device 10 can reduce breakage or pinching of the first end 16 of the breath collection device 10 while it is coupled to the hood member 150.

In another embodiment, air can be blown “upwards” through the device. That is, air (e.g., breath) can be blown into the breath collection device 10 through the second plug member 18 and out of the breath collection device 10 through the first plug member 14. By blowing air upwards through the device, the reagent can be percolated or moved around within the device so that the reagent can experience greater exposure to the blown air. If desired, a hood member (such as discussed above) or other air redirecting device could be used to redirect air upwards so that the user can blow air in a substantially horizontal direction and the air will be redirected appropriately into the device 10.

FIG. 5A is an isolated side view of the outer member 12 and FIG. 5B is an isolated top view of the outer member 12. As discussed above, the outer member 12 is preferably formed of a material that is sufficiently flexible to permit the transfer of a compressive force (such as manually exerted force) from the outer member 12 to a container 22 within the outer member 12. The outer member 12 is also preferably optically transparent so that incident light from the measuring device can be transmitted through the outer member 12 without significant distortion. The thickness of the outer member 12 can vary depending on the material selected for the outer member 12. However, the outer member 12 is preferably sufficiently thick to prevent broken pieces of container 22 from ripping, cutting, or otherwise penetrating the outer member 22. If the outer member 12 is formed without sufficient thickness or strength, broken pieces of container could penetrate the outer member, which could cause physical injury or distortion of the measurement taken by the measurement device.

On the other hand, to reduce the impact of the material on the transmission of light through the outer member 12, the outer member 12 is desirably formed with as thin a material as possible. In a preferred embodiment, the outer member 12 can be formed of propionate plastic that has minimal striations or other flaws in surface variation or quality that would impact the incident and reflected light associated with the measurement devices described herein.

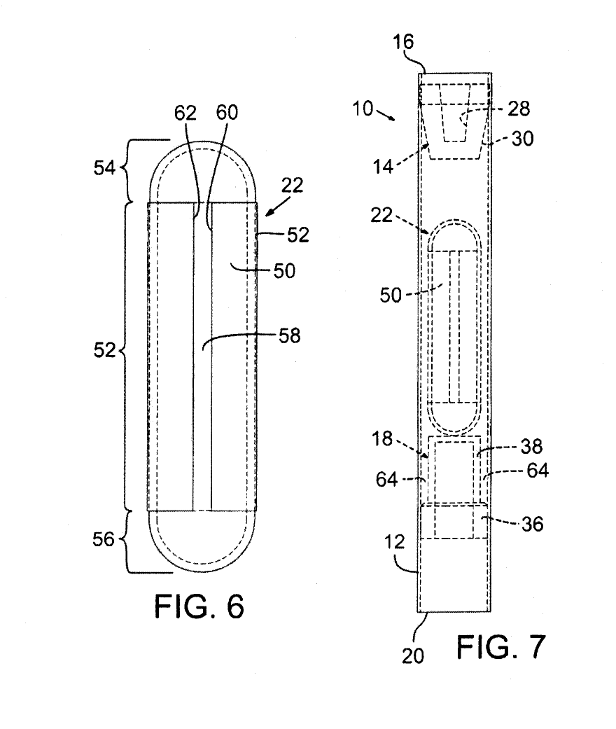

FIG. 6 is a side view of the container 22 configured to hold a reagent. As discussed above, in the illustrated embodiment, the container 22 can be a glass container (ampoule) that can be broken through the application of a compressive force applied to an outer surface of the outer member 12. For example, an individual can squeeze the outer member 12, applying sufficient compressive force to break the container 22 and release reagent particles from the container 22. The size and shape of the container 22 can vary depending on the amount and form of the reagent that it holds.

In a preferred embodiment, container 22 includes a wrap 50 that extends at least partially around the container 22. In the illustrated embodiment, the wrap 50 extends around approximately 90 percent (or about 320°) of the circumference of cylindrical portion 52 of the container 22, leaving cap portions 54, 56 and a gap portion 58 uncovered. The wrap 50 is configured to reduce the amount of broken container pieces (e.g., glass shards, etc.) and/or increase the size of the broken container pieces that are present in the outer member 12 after the container 22 is broken. Preferably, the wrap 50 is configured to capture and hold the broken container pieces in sizes that are of sufficient size that the broken container pieces are substantially prevented or restricted from entering into the measurement area 64 (FIG. 7).

For example, in a preferred embodiment, the wrap 50 is a plastic coating that covers between about 70 and 100 percent of the surface area of the cylindrical portion 52. More preferably the wrap 50 covers between about 85 and 95 percent of the surface area of the cylindrical portion 52, but does not circumscribe the entire circumference of cylindrical portion 52. In the illustrated embodiment, the wrap 50 has a first end 60 and a second end 62 that extend around the cylindrical portion 52 of the container 22. The first end 60 and second end 62 do not overlap and, therefore, result in the gap portion 58 being formed between the two ends of the wrap 50. The presence of the gap portion 58 makes it easier to break the container 22, since a wrap 50 that completely covers the surface of the cylindrical portion 52 results in a container 22 that is somewhat more difficult to break through the application of a manually exerted force.

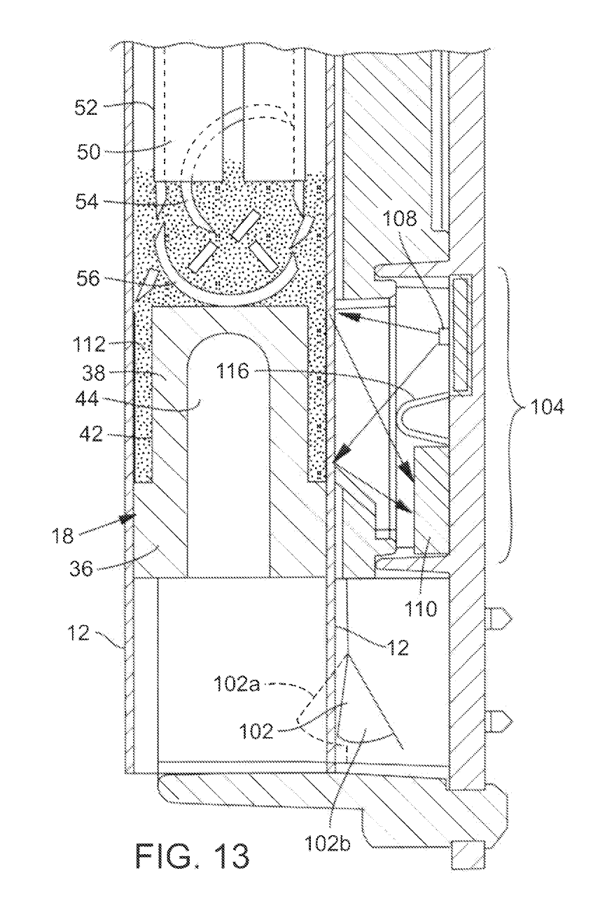

As shown in more detail below (FIG. 13), when a container 22 with a wrap 50 is broken, the container 22 essentially breaks into two cap portions 54, 56, some broken pieces of container from the gap portion 50, and a larger portion that is held together by wrap 50. Because of the size of these broken container pieces (especially the cap portions and the wrap-held portion), broken container pieces are substantially prevented from entering into the measurement area 64 (FIG. 7) and causing any distortion or other optical difficulties with regard to the measurement of the sample.

After the container 22 is broken, the reagent is no longer in an airtight container and ambient air can interact with the reagent. Thus, it is desirable to collect a breath sample shortly after the container 22 is broken. The time available to capture a breath sample depends on the reactivity of the reagent with the ambient air. Moreover, after the breath sample is collected in the breath collection device, it is desirable that the measurement of the sample by the measurement device occur within a relatively short time period in order to ensure that the measurement is accurate. Again, time available for taking the measurement can vary depending on the reagent that is being used.

The reagent contained in the container 22 is selected based on the substance in the exhaled breath that is to be detected. Particular reagents are discussed in more detail below. In addition, the reagent quantity can be selected based on the reactivity of the reagent to the substance in the exhaled breath that is to be measured. Moreover, the volume and pressure of breath (determined by blow time, volume of the outer member between plugs, plug porosity, etc.) can affect the amount of reagent necessary for an accurate measurement from the measurement devices described herein.

Various methods can be used to determine the volume of exhaled air provided by the test subject. For example, a breath bag can be provided for attachment to the breath collection device. A breath bag (also known as a blow bag) is a conventional device that can be attached to the breath collection device in such a manner that it expands while a user is blowing into the breath collection device. Once the breath bag is full, the user can stop blowing into the breath collection device. Alternatively, a spirometer can be provided to measure the volume of air exhaled by the test subject and/or a manometer can be provided to measure the air pressure exhaled by the subject. These methods can provide an indication that a sufficient volume of exhaled air has passed through the device to react with the reagent and provide an accurate test result.

FIG. 7 illustrates a side view of a breath measurement device 10 that has a container 22 held within an outer member 12 between two plug members 14, 18. As shown in FIG. 7, the container 22 can have a longitudinal length that is less than the distance between the two plug members 14, 18 and a width that is less than the distance between opposing inner surfaces of the outer member 12. In this manner, the container 22 can move freely within the outer member 12 between the two plug members 14, 18. Alternatively, the container 22 can be sized to restrict or partially restrict movement with the outer member 12. In either embodiment, the clearance between the container 22 and the outer member 12 preferably is sufficiently small that compressive force exerted on the outer member 12 is transmitted to the container 22 to selectively rupture the container 22.

Other sizes and shapes of outer members and containers are possible. For example, as described in more detail below, in the illustrated embodiment, since the reagent receiving area around the second plug member is the same in each axial orientation, the measurement device can take a measurement with the outer member positioned in any axial orientation within the measurement device.

In other embodiments, however, it may be desirable to form the outer member so that the breath collection device can only be received in the measurement device in a discrete number of orientations. For example, it may be desirable to form the outer member with a flat surface that faces the measurement device's optical components. A flat surface can provide better optical results, which may result in a better spectrometer reading. Thus, the outer member can be formed, for example, so that it has a rectangular or triangular-shape in cross section.

If constructed with a flat surface, it may also be desirable to construct the second plug member so that only one flat side of the outer member receives reagent between the second plug member and the outer member. Thus, the first and second plug members would need to be modified to fit in the outer member, and the second plug member may be modified so that when the outer member is received in the measuring device, only a limited number of axial orientations of the outer member results in an orientation where exposed reagent faces the measurement device's optical components.

### Measurement Device

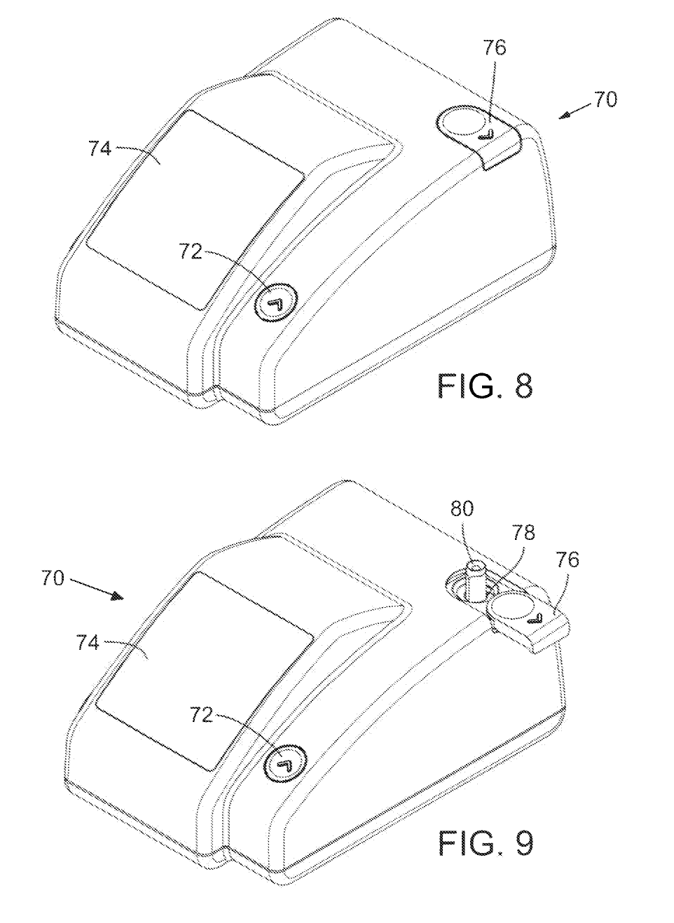

FIG. 8 is a perspective view of a measurement device 70 capable of receiving a breath collection device and taking a photometric reading (measurement) of a reagent that has been exposed to a breath sample. The device 70 can include a power switch 72, a display screen 74, and a sample receiving door 76. As shown in FIG. 9, sample receiving door 76 can be opened (e.g., by moving the door 76 laterally) to reveal a sample receiving area 78. When the door 76 is in the open position (FIG. 9), a sample 80 (e.g., a breath collection device 10 that has been exposed to an exhaled breath) can be inserted into the sample receiving area 78.

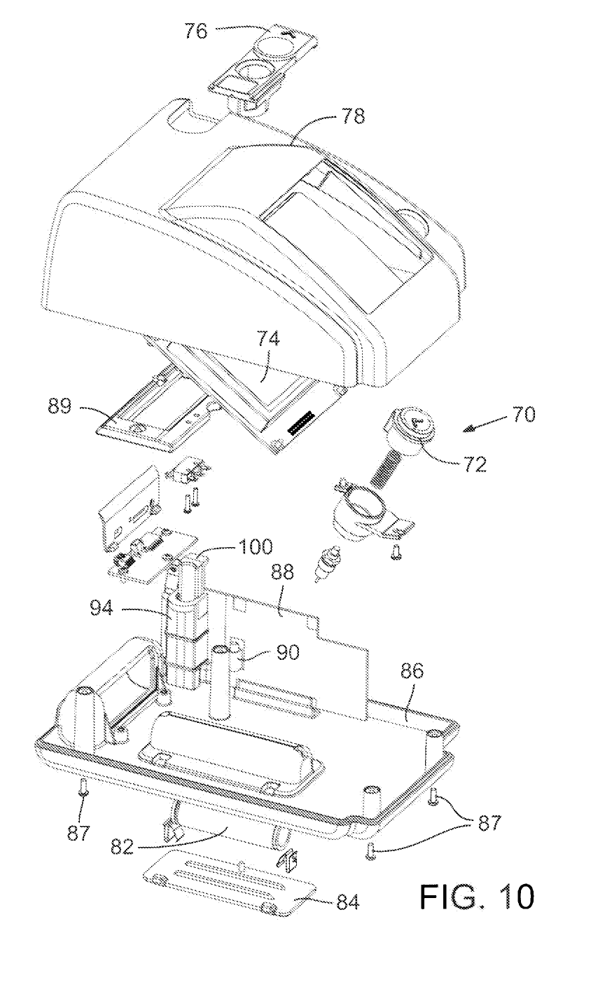

FIG. 10 illustrates an exploded view of the measurement device 70. In the illustrated embodiment, the measurement device includes a cover 78 and a bottom member 86 that can be attached to the cover with one or more fasteners, such as screws 87. Display screen 74 can also be attached to the cover 78 using one or more fasteners. The device 70 can be powered by a power source such as battery 82, which can be received in a lower portion of the bottom member 86 and covered by a battery door 84. If desired, battery 82 can be configured to be rechargeable. Alternatively, or in addition to battery 82, device 70 can be configured to operate on AC power.

Voltage and forward current from the power source is preferably controlled to minimize variations in LED strength during testing conditions. For example, a microprocessor can be configured to restrict voltage and current through the device unless certain conditions are met. In this manner, if the power source being used is batteries and the batteries are no longer sufficiently charged to power the LED sources at the desired levels, the microprocessor will prevent the measurement device from operating.

Door 76 can be coupled to door frame 89 in a manner so that door 76 is capable of being moved between a closed position (FIG. 8) and an open position (FIG. 9). FIG. 10 also shows a side wall 88 coupled to the bottom member 86.

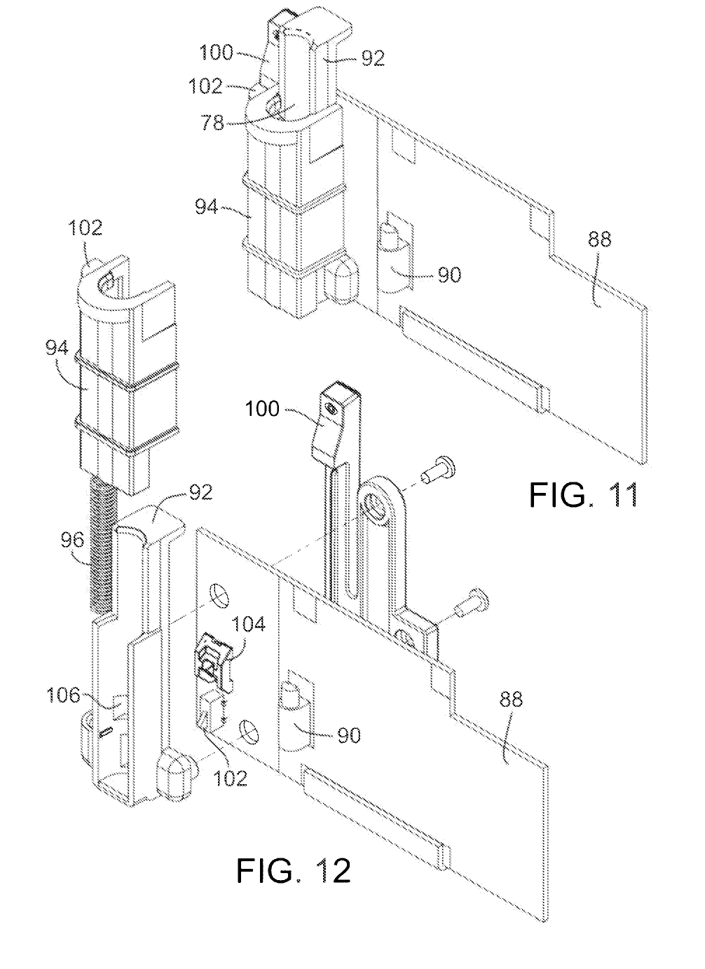

Referring to FIGS. 11 and 12, the side wall 88 is shown in more detail. The sample receiving area 78 is defined by a vertical member 92 and a vertical cover 94 (collectively, a chimney assembly). Vertical cover 94 is movable relative vertical member 92. A spring member 96 biases the vertical cover 94 upwards. To load a sample 80 into the sample receiving area 78, a sample 80 is pushed into the sample receiving area until a bottom portion of the sample 80 engages with a lip portion (not shown) on a lower portion of the vertical cover 94. A downward force can be directed at the top of the sample 80 causing the sample 80 and the vertical cover 94 to move downward together until the latch 100 catches on extending portion 102. The latch 100 catching against extending portion 102 locks the vertical cover 94 in place with the sample 80 in position to be analyzed by the device 70, as described below.

To release the sample 80 from the locked position, the latch 100 can be moved laterally. Referring to FIG. 10, the door 76 can have a portion that is configured to contact latch 100 when the door 76 moves from a closed position (FIG. 8) to an open position (FIG. 9). Thus, latch 100 is released by moving the door 76 to the open position and the sample 80 (and vertical cover 94) is forced upwards by spring member 96. The outer diameter of the sample 80 can be sized so that it has a loose friction fit with the sample receiving area so that, upon release, the kinetic energy of the spring will release the sample 80 with significant speed or force. In a preferred embodiment, the door 76 must be moved beyond a simple open position to a point where the latch 100 is activated to release the sample 80 from the locked position.

If desired, the door 76 can be configured so that when a sample 80 is inside the device 70, opening the door 76 does not fully release the sample. Instead, additional pressure must be applied to the door 76 to move the door 76 to a sample-releasing position (not shown), which is a position beyond the open position. The utilization of a sample-releasing position can further prevent the sample 80 from ejecting out of the device 70 in an uncontrolled manner. Instead, by moving the door 76 to the fully released position, the sample 80 can be gently released from the device 70.

The device 70 includes a light measurement system 104 positioned adjacent a window 106. The light measurement system 104 includes one or more light emitting devices (e.g., LEDs) and one or more light sensing devices (e.g., photometers). As discussed in more detail below, the light measurement system 104 can also define a light pathway between the light emitting device(s) and the light sensing device(s). The window 106 provides access to a portion of sample 80 so that the sample 80 can be exposed to light emitted from the light emitting device(s) of the light measurement system 104.

The device 70 can also include a switch 102 to identify whether a sample 80 has been positioned within the device 70. As shown in FIG. 13, the switch 102 is movable between a first position 102a and a second position 102b. When a sample 80 is not received in the device and locked into position by the latch 100, the switch 102 is in the first position 102a. As the sample moves into position within the device 70, however, the switch can move to the second position 102b, which sends a signal to the device 70 indicating that a sample 80 is in position and ready to be measured. A vibration device 90 can be positioned near the sample 80 (such as on the side wall 88), as shown in FIG. 10. The vibration device 90 can be configured to begin vibrating when a sample 80 triggers switch 102. The vibration of the vibration device 90 in turn vibrates sample 80 and causes the reagent 102 to settle within the outer member 12 between the outer member 12 and the outer surface 42 of the second plug member 18 to provide a more accurate reading.

Since the presence of broken glass or other contaminants in the measurement area can interfere with the light emitted and received by the light measurement system 104, the broken container pieces are preferably substantially prevented from entering the measurement area. As a result of the wrap 50, the broken portions of the reagent holding container 22 are substantially restricted to a first and second cap portion 54, 56, and a wrapped cylindrical portion 52. The size of the broken portions of the reagent holding container 22 are preferably too large to fit into the space between the second plug member 18 and the outer member 12 (i.e., measurement area 64 as shown in FIG. 7). Therefore, few, if any, broken pieces of container 22 are present in the measurement area of the sample 80.

Moreover, the vibration of the vibration device 90 will cause the reagent particles, which are typically smaller in size than any broken pieces of the container, to settle, forcing the broken pieces to the top of the reagent particles. This settling of reagent particles can further reduce the amount of broken pieces of container that are present in the area from which the light measurements are taken.

FIG. 13 illustrates an embodiment of a light measurement system 104. Light measurement system 104 includes a light emitting device 108 (e.g., LED) and a light receiving device (e.g., a photometer) 110. The light emitting device 108 and light receiving device 110 can be coupled to one or more circuit boards. After the light measurement system 104 takes one or more measurements as discussed below, an algorithm (described in more detail below) can be used to generate a quantitative “score” reflective of the amount of aldehydes detected by the device 70. If desired a qualitative “red/green” indicator can be used to identify the quantity of aldehydes in the breath. However, a numerical “score” is preferred so that the amount of aldehydes detected by the device 70 can be more accurately identified. The range of the “score” can be selected based on the accuracy with which the substance being measured can be identified. In most cases, a range of 1-100 or 1-1000 is sufficient.

The LED emission spectrum and photometer response spectrum can be selected based on the particular chemistry to be measured. Thus, for example, if desired, an LED that emits a relatively narrow spectrum of light can be used to direct a specific wavelength of light at an exposed reagent. Alternatively, an LED can be selected that delivers a broader spectrum of light (e.g., a white light) at the exposed reagent. Similarly, different photometers can be selected depending on the breadth of the spectrum of light that is relevant to the colorimetric reaction that is to be measured.

As shown by the arrows, light is directed from the LED 108 through the window 106, through the portion of the outer member 12 that is exposed by the window, to the reagent 112. At least a portion of the light that is not absorbed by the reagent 112 is reflected back to the photometer 110. An intermediate element (light directing/blocking element) 116 is positioned between the LED 108 and the photometer 110 to restrict light emitted from the LED 108 from directly striking photometer 110 without first being incident on the exposed reagent portion.

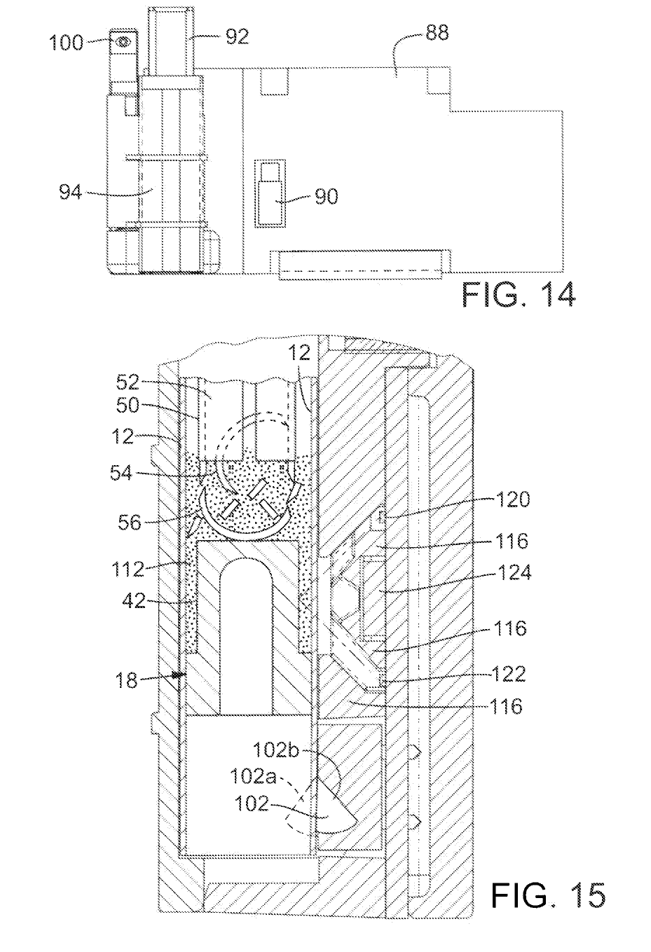

FIGS. 14 and 15 illustrate another embodiment of a light measurement system. As shown in FIG. 15, the light measurement system of this embodiment comprises a plurality of LEDs (first LED 120 and second LED 122) and at least a one photometer (124). The light measurement system of FIG. 15 further includes intermediate elements (light directing/blocking elements) 116 positioned adjacent the LEDs 120, 122 to guide light from the LEDs 120, 122 to the exposed reagent that is to be measured. The positioning of the intermediate elements 116 reduces the effective aperture of the LEDs 120, 122, reducing the likelihood that aberrant light from the LEDs 120, 122 will strike a surface other than the desired portion of the outer member 12 that contains reagent particles 112.

In addition, it can be advantageous to position the photometer (light receiving device) 124 so that it is in a direct vertical line of sight of the reagent particles 112, as shown in FIG. 15. In other words, it is advantageous to arrange or position the photometer 124 so that light directed at an angle of approximately 90° from at least some of the reagent particles will be received by the photometer 124. By arranging the photometer 124 in this manner, the impact of light reflecting off of the outer tube (or other non-reagent material) can be reduced. Additionally, a greater quantity of light reflected from the reagent may be received by the photometer 124.

First and second LEDs can be used to emit the same spectrum of light or they can be used to emit different spectrums of light. If the first and second LEDs are configured to emit the same spectrum of light, the amount of light emitted at the exposed reagent is doubled, providing a greater amount of reflected light that can be measured by the photometer. In other embodiments, however, it may be desirable to configure the first LED to emit a first spectrum of light and the second LED to emit a second spectrum of light that is different from the first spectrum of light. Thus, the first and second LEDs can measure the colorimetric reactivity of a reagent at two different spectrum regions.

Various wavelength filters can be used in connection with the devices disclosed herein. For example, the effective wavelength(s) that the photometer system measures can be modified as needed by the addition of wide band optical filters on either the emitting (LED) side and/or the receiving (photometer) side of the system. Thus, if desired, the infrared (UV) range from 620 nm to longer wavelengths and the ultraviolet range from approximately 350 nm and shorter wavelengths can be restricted using a wavelength filter. Additionally, narrow band optical filters can be used to limit noise and optimize signal in the areas of maximum reaction to the breath sample(s).

Preferably, the surfaces in the measurement area of the measurement device are configured to be non-reflective to reduce reflectance from non-sample surfaces. Thus, to the extent possible, all surfaces within the measurement area are desirably colored black or otherwise rendered non-reflective to reduce undesirable internal reflections at or about the measurement area. For example, the surfaces of the plug member and circuit boards located near the measurement area are preferably black to reduce any unwanted reflections from those surfaces. In addition, the measurement device is preferably “light-tight” in the vicinity of the measurement area. That is, external light is substantially or completely restricted from entering the measurement device and causing distortion of the readings taken at the measurement area.

In some embodiments, it may be desirable to take a reading of the reagent before exposing the reagent to a breath sample. Thus, the reagent container (ampoule) can be broken and the outer member can be placed into the measurement device to obtain a light measurement reading for the unexposed reagent. Such a reading can be helpful to establish a baseline reading associated with the reagent, which can help account for minor variations in reagent chemistry between batches and/or account for possible inconsistencies in the readings provided by the measurement device itself

### Reagents

The breath collection devices and the measurement devices described herein can be used in combination with a variety of reagents to detect various substances that are present in the exhaled breath of a test subject. For example, both volatile and non-volatile compounds, such as select chemicals (e.g., ammonia, urea) small molecules (e.g., nitric oxide), and protein and peptides (e.g., cytokines) can be detected. Such devices and reagents can be used for detection of various conditions or states including, for example, illnesses or other physical conditions, and indicators of illegal or legal drug use (e.g., alcohol or other drug screening).

In one embodiment, the reagent can comprise a reagent for determining a quantity of ethyl alcohol in a breath sample. For example, reagents such as those described in U.S. Pat. No. 4,105,409 to Monnier et al., the entire disclosure of which is incorporated herein by reference, can be used. As described in the '409 patent, the reagent can consists of an mixture of (1) iodine pentoxide, (2) a colorless metal nitrate or concentrated nitric acid and (3) 75 to 98% (wt./wt.) sulphuric acid. A color reaction occurs when ethyl alcohol is exposed to the reagent, changing the white color of the reagent to pink, brown or black depending on the quantity of ethyl alcohol that is added.

In a preferred embodiment, the sulphuric acid concentration lies between 80 and 90% (wt./wt.), and sodium nitrate, potassium nitrate or cerium(III) nitrate hexahydrate, Ce(NO₃)₃.6H₂O is used as the colorless metal nitrate. The reagent can be conveniently adsorbed on a solid, inert, porous carrier and used in this form. Suitable carriers are for instance silica gel, kieselguhr (diatomaceous earth), fuller's earth, zeolites and aluminium oxide. Silica gel is preferred, particularly one with an average grain size of 0.2 to 0.5 mm (equivalent to 35 to 70 mesh according to ASTM), e.g. “Kieselgel 100” made by Merck AG, Darmstadt (W. Germany). The breath to be investigated can be exhaled through the porous reagent mass.

In a preferred embodiment of the invention, for each 100 g of silica gel, the reagent contains (1) 10 to 50 g, or more preferably 10 to 20 g, of iodine pentoxide, (2) 5 to 25 g, or more preferably 5 to 15 g, of metal nitrate or 3 to 10 ml, or more preferably 4 to 5 ml of concentrated nitric acid, and (3) 50 to 120 but preferably 80 to 100 ml of 80 to 98% (wt./wt.) sulphuric acid. In a specific embodiment, the reagent can be formed as described in Example 1 below.

### Example 1

100 parts of “Kieselgel 100” silica gel made by Merck AG, previously well dried at 110 degrees C., 184 parts (=100 parts by vol.) of 98% sulphuric acid, 15 parts of iodine pentoxide and 5 parts of cerium(III) nitrate hexahydrate, Ce(NO₃)₃.6H₂O, are used.

The silica gel is slowly impregnated, with stirring, with the sulphuric acid to give a completely homogeneous mixture. The finely ground iodine pentoxide and the finely ground cerium(III) nitrate hexahydrate are mixed well together and this mixture added gradually to the impregnated silica gel while the latter is still pasty and in any case before it has dried out completely. The resulting product is then rigorously mixed and reduced in size in a shaking machine until a fine, solid granulate material is formed.

A given quantity of the granular material is placed in 5 cm long tubes and compacted to fill a length of 1 cm in the middle of the tube. The reagent mass is held in place between two air-permeable supports. Suitable supports are sintered glass discs, plugs of glass wool or rectangular Teflon rods. Both ends of the tube are then sealed by melting. Care should be taken that the tube and supports are clean and that the tube is not sealed too close to the reagent since the reagent becomes colored and thus unusable under the influence of heat.

In another example for detecting ethyl alcohol, a granular color indicator can be provided as disclosed in U.S. Pat. No. 5,834,626 to De Castro et al., the entire disclosure of which is incorporated by reference herein. An example of such a granular indicator is described below in Example 2.

### Example 2

Prepare granular solid support by mixing 27 grams of 70-230 mesh silica gel (American Scientific Products, IL) with 200 mL D/I water, and 40 mL concentrated nitric acid. Stir at room temperature overnight. Filter, rinse with D/I water, and vacuum dry. Prepare a 0.2 L of a solution of 1M potassium dichromate (K 2 Cr 2 O 7) in 1M sulfuric acid (H 2 SO 4). Mix pretreated support with the potassium dichromate/acid solution overnight. Filter, rinse extensively. Dry in vacuum oven at 40° C. for 4 hours.

Pack granular support into the interstitial space of a tube assembly, or immobilize onto a strip comprised of an inert plastic film and an adhesive. For the case of a strip (5×0.7 cm) approximately 0.1 grams of indicator is immobilized, as measured via an electronic balance. Insert the strip inside the middle of a testing tube 10 cm long by 1 cm diameter.

Various levels of alcohol vapor are readily introduced into the device by mixing fixed amounts of ethanol with water, rinsing and gargling for at least 5 minutes, and exhaling into a tube connected to the volume-measuring device described previously.

In another embodiment, a reagent is provided that is reactive with one or more aldehydes in a test subject's breath. These aldehydes are byproducts of and are directly correlated with oxidative stress, along with associated health risks. The measured aldehydes are critical biomarkers of lipid peroxidation, the process by which excess free radicals attack lipids in cell membranes causing tissue damage.

The reagent colorimetrically reacts rapidly with many aldehydes (saturated and unsaturated) associated with oxidative stress including, but not limited to, hexanal, heptanal, decanal, and MDA. The colorimetric reaction to the cumulative aldehydes present is then measured by a measurement device, which generates a result based on the intensity of the reaction as determined by the algorithm described below. In particular, the reagent will experience a change in color spectrum relative to the amount of aldehydes present in the sample. If aldehydes are present, the reagent will produce a color change commensurate with the concentration of aldehydes present. Because other elements or compounds can result in color change to the reagent (e.g., the presence of moisture), the device is configured to determine particular color changes that are indicators of the aldehyde's presence.

In a preferred embodiment, the reagent is a reagent capable of indicating the presence of free radicals (hereinafter “FR reagent”) which is a solid phase reagent that is stored in an airtight container (such as the container 22, described above) and released from the container in anticipation of exposure to the breath of a test subject. The FR reagent can be, for example, a powder composed of porous silica gel to which a reactive component, such as a Schiff reagent, is absorbed and retained.

Schiff reagents are solutions that are known to chemically react to the presence of aldehydes by exhibiting a color change to a magenta or purple color. As a result, Schiff reagents in liquid phase are routinely used in tissue staining procedures. However, liquid phase reagents are generally less suitable for use with a breath collection device that requires one or more porous plug members for receiving an exhaled breath sample. A liquid would be prone to leak through the porous plug membranes. Accordingly, methods for forming FR reagents in solid phase are provided.

Schiff reagents are generally formed by the reaction of pararosaniline or rosaniline with sodium bisulfite. Although pararosaniline has generally been considered much more suitable for creating reagents that are capable of detecting aldehydes, Applicants have found that rosaniline works surprisingly well as a reactive component of an FR reagent in the solid phase. Accordingly, in a preferred embodiment, rosaniline hydrochloride (fuchsine) is the reactive component present in the FR reagent. Various other dyes can be Schiff reagents, as that term is used herein, including derivatives of and/or chemical modifications of pararosaniline and rosaniline can also be used. Such useful dyes can include, for example, rosaniline having a single methyl group (Basic Fuchsin), rosaniline having a dimethyl group (Magenta II), and rosaniline having a trimethyl group (New Fuchsin).

Initially, the liquid phase Schiff reagent can be formed using conventional methods. For example, rosaniline can be converted to a Schiff reagent by combining a quantity of rosaniline with sodium metabisulfite. In acidic aqueous solution, sodium metabisulfite produces sulfurous acid, which adds a sulphonate group to the central carbon of rosaniline, which decolorizes the Schiff reagent. If desired, charcoal can be added to the solution to remove impurities. Later, the charcoal can be removed by filtration to decolorize the Schiff reagent. Also, phosphoric acid can be added to stabilize the pH of the solution.

The FR reagent can include a silica gel that adsorbs the Schiff reagent. Preferably, the silica gel is pretreated with an acid solution, preferably phosphoric acid, in order to lower the pH of the silica and prepare it for receiving the Schiff reagent. The pH is preferably lowered by at least about 0.2 pH, more preferably lowered at least about 0.4 pH, and even more preferably lowered at least about 0.6 pH. If the silica gel pH is lowered by adding solution (e.g., an acidic solution), the silica gel solution can be heated to substantially dehydrate the solution to return the mixture to a solid phase. For example, the silica gel can be mixed with a phosphoric acid solution at about a 1:2 ratio and oven dried at about 60 to 100 degrees Celsius, more preferably about 70 to 80 degrees Celsius, to approximately 10% of the initial silica weight.

The pretreated silica and the Schiff reagent can then be combined to form the FR reagent. Preferably, the pretreated silica is mixed with a diluted Schiff reagent at about a 1:2 ratio and then oven dried to at least substantially dehydrate the mixture. The mixture is preferably dried to the approximate initial weight of the silica. Preferably, the oven drying step occurs at about 80 degrees Celsius or lower. Instead of including an oven drying step, the mixture can be dried or dehydrated using other methods including, for example, chemical drying and/or lyophilization. An embodiment of the process described above is set out in Example 3 below in more detail.

### Example 3

Schiff Reagent Formulation

1. Dissolve sodium metabisulfite (in water).

2. Add basic fuchsine to the metabisulfite solution and mix until dissolved (about 10 minutes).

3. Add charcoal to the solution, mix for about 30 minutes.

4. Allow mixture to incubate at ambient temperature for at least 24 hours, but less than 36 hours.

5. Filter the solution to remove the charcoal.

6. Adjust the pH of the solution from about 2.5±0.4 to 1.88±0.05 with 75% phosphoric acid.

7. Add small amount of de-ionized water to complete total batch size and mix until solubilized.

8. Store the solution in a glass container and seal with paraffin.

9. Store container in a cool, dry place until use.

Silica Gel Preparation

1. Acidify 644 silica with 3.75% phosphoric acid at a ratio of 1:2 by weight.

2. Mix until homogenous blend is achieved (about 5 minutes).

3. Dry at about 80 degrees Celsius (±5 degrees) to about original silica weight (±about 5%).

4. Cap with argon and seal with paraffin.

5. Store at room temperature until use.

FR Reagent Preparation

1. Combine 2 parts Schiff reagent with 1 part dry, acified 644 Silica.

2. Mix for about 5 minutes.

3. Dry to about the original weight of the silica, check hourly. Preferably, the drying takes place about 80 degrees Celsius or lower.

4. Cap with argon.

5. Store in sealed jar at room temperature.

The solid phase FR reagent preferably captures the gaseous and vaporized phase of breath, not solely exhaled breath condensate (EBC). Accordingly, a breath collection device containing FR reagent can collect a breath sample and the measurement of that sample by a measurement device provides a substantially real time capture of breath, not a capture of a fluid or sample for subsequent analysis in a laboratory.

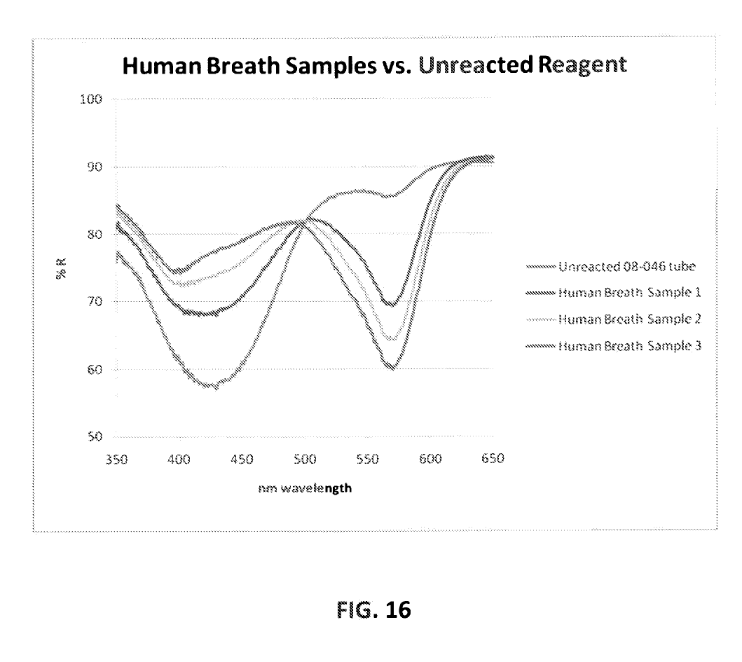

The solid phase FR reagent appears to reacts differently than basic fuchsine to the presence of aldehydes. Generally, the color change associated with Schiff reactions occurs at a wavelength of about 570 nm. However, as shown in the graph of FIG. 16, exposure of the FR reagent to exhaled breath produces two areas of interest: one at about 440 nm and another at about 570 nm. Without being bound by theory, it is believed that the reactivity in the area of the longer wavelength region is more highly associated with the amount of moisture in the breath of a test subject that is the area in the shorter wavelength.

FIG. 16 is a graph of percent reflectance as a function of wavelength (nm). FIG. 16 depicts four different curves. The first curve is designated as “Unreacted 08-046 tube” and identified on the graph as the curve with the lowest level of percent reflectance in the region between about 400-450 nm. The first curve represents the reflectance of an unreacted sample of reagent in a tube. The second curve is “Human Breath Sample 1” and can be identified on the graph as the curve with the second lowest level of percent reflectance in the region between about 400-450 nm. The third curve is “Human Breath Sample 2” and can be identified on the graph as the curve with the third lowest level of percent reflectance in the region between about 400-450 nm. The fourth curve is “Human Breath Sample 3” and can be identified on the graph as the curve with the highest level of percent reflectance in the region between about 400-450 nm.

As seen from FIG. 16, two significant changes in reflectance occur between about 350 nm and 650 nm upon exposure to a breath sample. The first is an increase in percent reflectance upon exposure to a breath sample in the region between about 400 nm and 450 nm. For example, each of the curves that correspond to a breath sample reflects a higher level of reflectance in the region between about 400 nm and 450 nm relative to the curve of the unreacted reagent sample. The second is a decrease in percent reflectance in the region between about 550 nm and 600 nm upon exposure to a breath sample. For example, each of the curves that correspond to a breath sample reflects a lower level of reflectance in the region between about 550 nm and 600 nm relative to the curve of the unreacted reagent sample. It is in these wavelength ranges that the FR reagent exhibits significant colorimetric reactivity to breath, including aldehydes in the breath. Accordingly, the light measurement device described above, preferably at least measures the colorimetric reactivity of the FR reagent in the region between about 350 nm and 650 nm. More preferably, the light measurement device is configured to measure at least two regions of reactivity, such as a first region of about 400-450 nm and a second region of about 550-600 nm.

Without being bound by theory, it is believed that the FR reagent reacts to aldehydes at both regions, but to a greater degree in the area of between about 550 nm and 600 nm. In addition, while it is believed that the FR reagent reacts to moisture in the breath at both regions, the measurement of the reactivity of the FR reagent in the area of between about 400 nm and 450 nm (e.g., about 440 nm) is believed to more significantly correspond to the moisture reactivity of the Schiff reagent. The term “moisture” or “breath moisture,” as used herein, refers to the portion of the breath that is not being specifically measured by the selected reagent. For example, in this embodiment, moisture refers to anything that is not the aldehydes being measured by the FR reagent.

As described in more detail below, the moisture reactivity can be used to normalize a score (or measurement) obtained using the measurement device. Alternatively, a moisture reactive chemical can be additionally added to the FR reagent to provide an additional means for quantifying the amount of moisture present in the sample from the breath. As discussed in more detail below, an algorithm can be configured to take into consideration the moisture reactivity of the additional chemical or deconvolution analysis of the 400 nm-450 nm region to normalize the measurements of aldehyde reactions in breath samples.

It has been found that the amount of time after exposure to breath can be significant in determining the amount of aldehydes (or other indicators) present in the breath sample. In particular, after exposure to a breath sample, the two regions, 400 nm-500 nm and 550 nm-600 nm, experience changes in reflectance that are different from one another over time. That is, the regions vary over time with different rates of reflectance change because the reactions occur at different speeds. Accordingly, it can be desirable to take multiple reflectance measurements at the above two regions. In addition, when determining the amount of aldehydes (or other indicators), it can be useful to take into consideration the time after exposure to the breath sample in which the measurements were taken, as well as the relative changes in reflectance at the two regions at the time the measurements were taken. By taking a plurality of measurements for at least the two regions, a rate of change of reflectance for both regions can be determined.

In view of the different rates of change in reflectance over time between the two regions, it is desirable to take a plurality of reflectance measurements (or other similar light measurements) over time from which the amount of aldehydes can be determined. The selection of which measurement(s) should be used to calculate the amount of aldehydes (or other indicators) in the breath sample can be determined based on the respective rates of change of the two regions.

An algorithm, as discussed in more detail below, can be used to select reflectance measurements that are based on the most accurate points to measure the amount of aldehydes. In some embodiments, the most accurate points of measurement (in time) may be when the rate of change of reflectance reaches a predetermined level. Thus, a determination of the relative rates of change in the amount of reflectance measured at two or more regions can provide a more accurate means to measure the amount of aldehydes (or other indicators) present in a breath sample.

Schiff reagents are conventionally only available in solution because Schiff reagents are unstable and readily release the sulphonate group at the central carbon reforming rosaniline or pararosaniline. For this reason, sufficient amounts of sulfurous agents are required in the solution to stabilize and maintain the Schiff reagent. Surprisingly, it was found that the resulting solid phase FR reagent retains sufficient Schiff reagent to provide the requisite color change upon exposure to exhaled breath.

Although the illustrated embodiment of FR reagent uses a silica gel to provide the solid phase reagent, it should be understood that other solid surfaces may be used to hold various reagents. For example, liquid phase reagents can be adsorbed on other gels, papers, filters, or other surfaces using thin layer chromatography.

### Algorithm

Various algorithms can be used to quantify data obtained by the measurement devices described herein. The complexity of the algorithm will depend on the complexity of the function being measured.

Thus, in one embodiment, the function being measured can be quantified, or at least approximated, by a linear function. For example, in certain embodiments, a white light LED can be used to emit a broad spectrum of light at an exposed amount of FR reagent. A photometer can be configured to determine the amount of “red” light, “green” light, “blue” light, and “clear” light, with clear light being the total amount of light measured by the photometer. An exemplary linear function comprises calculating a quantitative “score” based the linear combination of one or more of (1) the ratio of measured red light to measured clear light (e.g., the amount of measured red light divided by the total amount of measured light), (2) the ratio of measured green light to measured clear light (e.g., the amount of measured green light divided by the total amount of measured light), and (3) the ratio of measured blue light to measured clear light (e.g., the amount of measured blue light divided by the total amount of measured light). If desirable, the linear function can be configured such that one or more areas of the measured spectrum are more heavily weighted than other areas of the measured spectrum.

The algorithm used to derive a quantitative “score” from one or more spectrometer measurements can also be more complex to provide a more accurate quantitative “score.” For example, the algorithm can be based on a rational or arbitrary function.

In another embodiment, an algorithm using an arbitrary function is provided. The algorithm can map a plurality of reflectance spectrum accumulated as three numbers to a score. For example, the three or more numbers can be representative of three or more different measured channels or portions of a light spectrum, such as channels including R, G, B, and clear (total) light. As the reflectance spectrum of FR reagent to aldehyde exposure does not appear to be an entirely linear function, an arbitrary function, together with a method for determining it, can provide improved accuracy and minimal variation of scores over a large number of samples.

Determining the arbitrary function amounts to assigning values to a grid of cells covering the 3 dimensional space of the three channels or portions, such as the R, G, B channels. This assignment of values converts to a problem of linear algebra, which can be solved using conventional methods. Two variants exist: one assigns values to cells to minimize the discrepancy between the score and aldehyde breath concentrations measured in a group of test subjects. Accordingly, this variant can be modified as additional data points concerning aldehyde breath concentrations are established. The other variant assigns values to cells to maximize the signal to noise ratio which is the ratio of the inter-personal variation in score with the average intra-personnel score.

The algorithm is also capable of normalizing the measurement of comparable concentrations of aldehydes by evaluating the amount of moisture content present in the measured sample. The amount of moisture in the breath is indicative of the volume of the breath exposed to the FR reagent. In addition, the amount of moisture in the breath is important to the reactivity of the FR reagent. Accordingly, a measurement of the amount of moisture in the breath can be used to normalize the measurement of the aldehydes among multiple samples and multiple test subjects.

As noted above, the solid phase FR reagent appears to reacts differently than basic fuchsine to the presence of aldehydes. Without being bound by theory, it is believed that the reactivity in the area of about 440 nm is largely associated with the amount of moisture in the breath of a test subject. Thus, the greater the amount of moisture in the breath, the greater the reflectance of light in the range or area of about 440 nm. Thus, a measurement of light reflectance (or absorption) exhibited in the area of about 400-450 nm can be used to normalize the measurement of aldehydes taken by the measurement device. Additionally, pure Schiff reacting with aldehydes can react in the 570 nm region and may require deconvoluting mathematically to distinguish signals.

### Operation of Measurement Device/User Interface

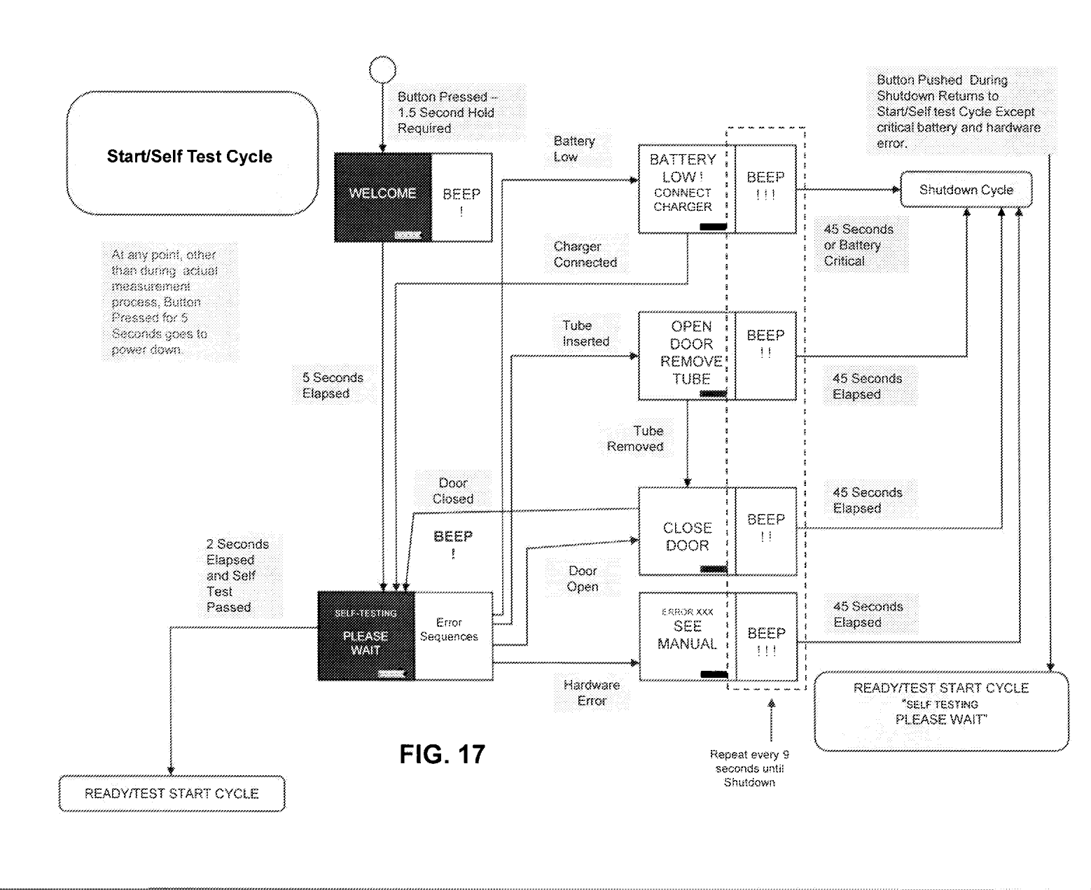

FIGS. 17-20 are flowcharts indicating the user interface and operations of an embodiment of a measuring device. FIG. 17 illustrates a “Ready/Test Start Cycle” whereby the measurement device is powered on and various system errors and/or non-functional configurations are identified. For example, after the measurement device is powered on, the device can cycle through a plurality of system checks to determine (1) whether the battery is low, (2) whether a previously tested sample is inserted in the device, (3) whether a sample-receiving door is open, and (4) whether there are any hardware errors. If any of these errors are present and remain uncorrected for a certain time period, the device turns itself off.

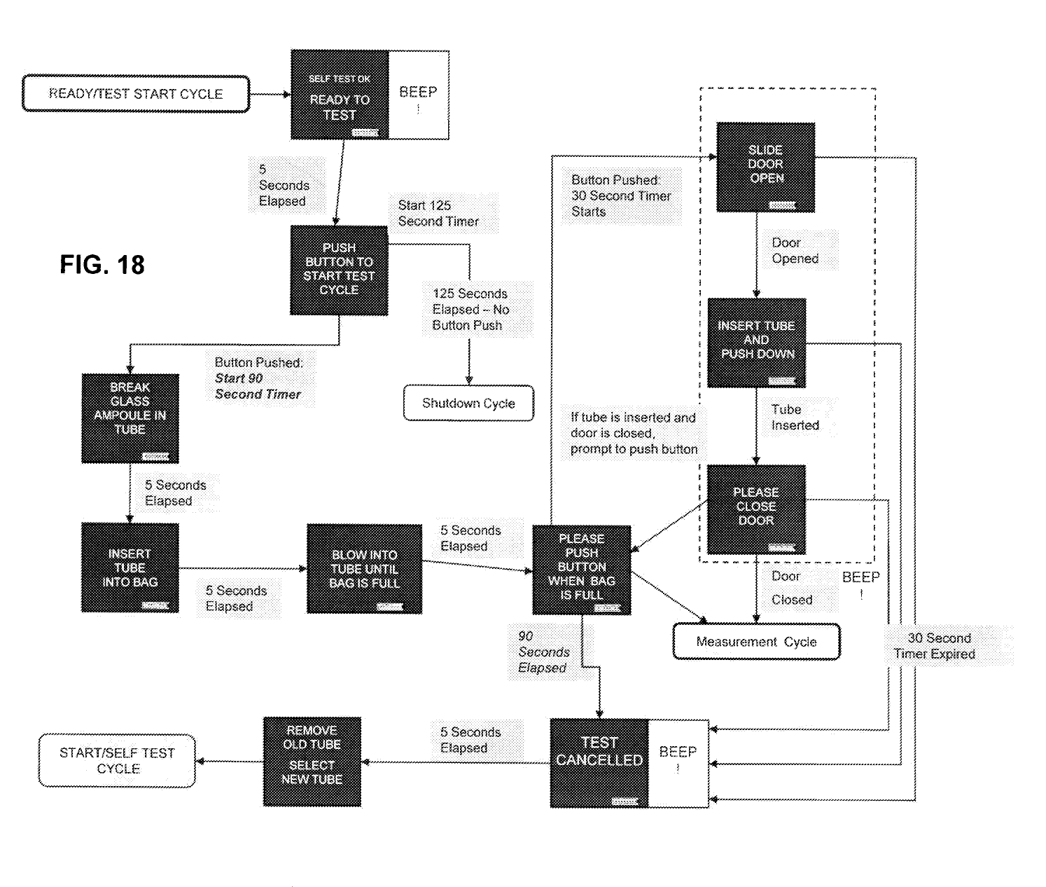

FIG. 18 illustrates a “Ready/Test Start Cycle” whereby the device has been powered on and no errors have been identified. The test cycle can be initiated by pressing the button to “Start Test Cycle.” Pressing this button can also trigger a timer. As discussed above, breath is preferably collected shortly after the reagent in the container is released and the collected sample is preferably measured shortly after the sample is collected. Accordingly, the timer can provide guidance to the user as to the amount of time that has lapsed since initiating the test. The device instructs the user to release the reagent (e.g., “Break Glass Ampoule in Tube”). Next the device instructs the user to insert the tube into a breath bag. If no breath bag is to be used, this step can be omitted.

Once the user has finished blowing into the breath collection device, the user presses a button “Please Push Button When Bag is Full.” If the user has exceeded the predetermined amount of time available for providing a breath sample (e.g., 90 seconds in the embodiment shown in FIG. 18), the test is canceled. If the user has not exceeded the predetermined amount of time, the user is instructed to open the door of the measurement device, place the sample into the measurement device, and close the door. Throughout these steps, if the user exceeds additional predetermined time limits, the test is canceled. Each of these predetermined time limits is provided to prevent testing of samples that are not suitable for testing because the user waited too long between releasing the reagent and collecting the breath sample, or between collecting the breath sample and testing the collected sample.

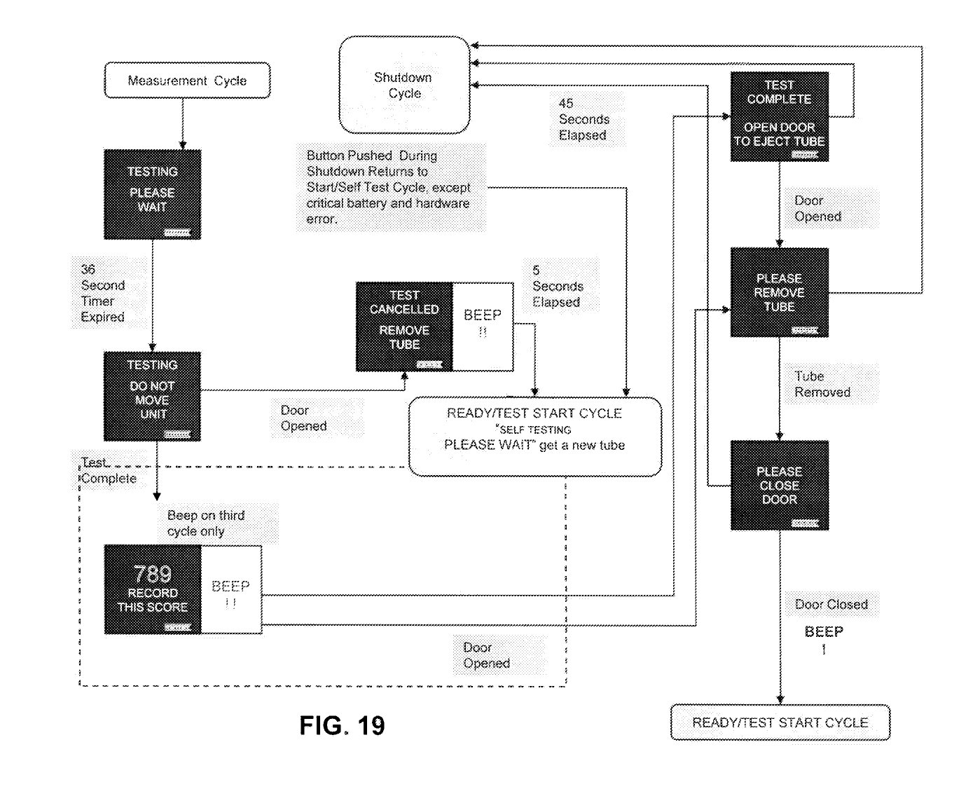

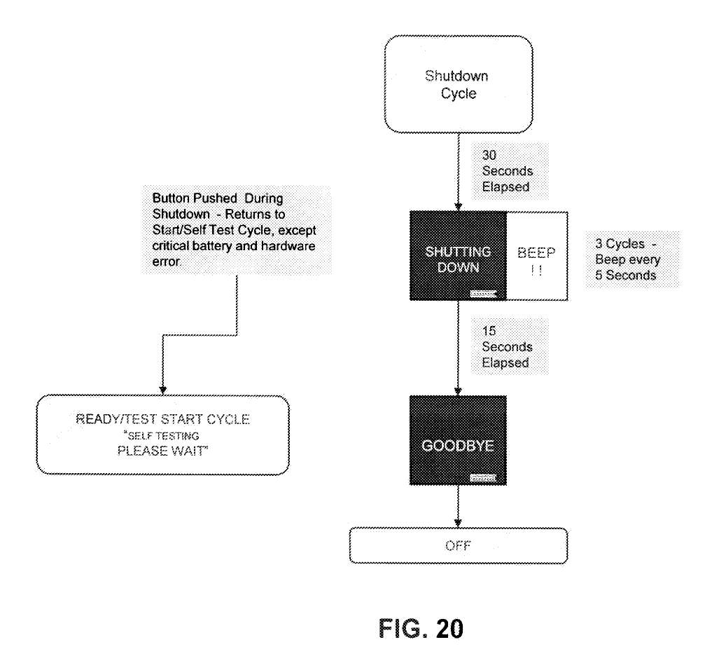

FIG. 19 illustrates an operation of the measurement cycle after a test sample has been placed in the device for testing. If the test is performed without interruption, the measurement device provides a test “score,” which is a quantitative value determined based on the readings from the photometer and the results of running those readings through the algorithm provided. After the score is displayed the user is instructed to open the door to eject the sample from the measurement device. After the sample is removed from the device, the measurement device can be shut down. FIG. 20 illustrates an operation for shutting down the system.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. An apparatus for measuring a quantity of an analyte contained in a breath sample, the apparatus comprising:
a breath collection device comprising a breath inlet area, a breath outlet area, and a reaction chamber, the reaction chamber including a reagent that is colorimetrically reactive with the analyte; and
a measurement device comprising a light emitting device and a light measuring device, the measurement device being configured to receive the breath collection device so that the reagent is positioned to receive light from the light emitting device and reflect at least a portion of the received light to the light measuring device.

2. The apparatus of claim 1, wherein the analyte comprises one or more aldehydes.

3. The apparatus of claim 1, wherein the light emitting device comprises a plurality of LEDs.

4. The apparatus of claim 3, wherein each of the plurality of LEDs emits light at the same wavelength.

5. The apparatus of claim 3, wherein each of the plurality of LEDs emits light at a different wavelength.

6. The apparatus of claim 5, comprising:
a first LED that emits light in a range absorbed by the reagent that corresponds, at least in part, to a reactivity of the reagent to moisture; and
a second LED that emits light in a range absorbed by the reagent that corresponds, at least in part, to a reactivity of the reagent to the analyte.

7. The apparatus of 1, further comprising a value generating device comprising an algorithm that generates a quantitative value based on one or colorimetric measurements taken by the measurement device on the reagent.

8. The apparatus of 7, further comprising a moisture reactivity measuring device, wherein the algorithm is configured to normalize the colorimetric measurements based on a measurement of an amount of moisture contained in the reagent.

9. The apparatus of claim 8, wherein the moisture reactivity measuring device comprises an LED that emits light in a range absorbed by the reagent that corresponds, at least in part, to a reactivity of the reagent to moisture.

10. A breath collection device comprising:
a tubular outer member having a first end portion and a second end portion,
a breath inlet area having a first porous plug member positioned in the tubular outer member at the first end portion;
a breath outlet area having a second porous plug member positioned in the tubular outer member at the second end portion, the second porous plug member having a first cylindrical portion with a first diameter and a second cylindrical portion with a second diameter, the second diameter being smaller than the first diameter; and
a reaction chamber within the tubular outer member and located between the first and second porous plug members, the reaction chamber including a reagent that is colorimetrically reactive with one or more substances contained in the exhaled breath of a test subject,
wherein the second cylindrical portion extends from the first cylindrical portion towards the first porous plug member.

11. The device of claim 10, further comprising a breakable container, the reagent being at least initially held within the container.

12. The device of claim 11, wherein the container comprises a wrap extending around at least a portion of the container to reduce the amount of broken pieces of container that result when the container is broken and the reagent released.

13. The device of claim 12, wherein the wrap extends around at least 70% of the container.

14. The device of claim 13, wherein the wrap extends around between about 85% and 95% of the container.

15. The device of claim 14, further comprising a hood member, the hood member comprising a breath receiving portion and a breath outflow portion,
wherein the breath outflow portion is configured to be removably coupled to the first end portion of the tubular outer member.

16. The device of claim 15, wherein the breath receiving portion and the breath outflow portion are configured at an angle of about 70 and 110 degrees relative to one another.

17. An apparatus for measuring a quantity of an analyte contained in a breath sample, the apparatus comprising:
a breath collection device comprising a breath inlet area, a breath outlet area, and a reaction chamber, the reaction chamber including a reagent that is colorimetrically reactive with the analyte;
a measurement device comprising a light emitting device and a light measuring device, the measurement device being configured to receive the breath collection device so that the reagent is positioned to receive light from the light emitting device and reflect at least a portion of the received light to the light measuring device, the light measuring device being configured to take a plurality of reflectance measurements over a predetermined time period for at least two wavelength regions; and
a measurement selection device configured to select one or more measurements taken by the measurement device to determine the quantity of the analyte, the measurement selection device being configured to select the one or more measurements based on a determination of a rate of change of reflectance of at least one of the at least two wavelength regions.

18. The apparatus of claim 17, wherein the at least two wavelength regions comprise a first region including wavelengths between about 400 nm and 450 nm and a second region including wavelengths between about 550 nm and 600 nm.

19. The apparatus of claim 18, wherein the rate of change of reflectance is determined in the second region.
